(19) United States
(12) Patent Application Publication
(10) Pub. No.: US 2009/0203941 A1
(43) Pub. Date: Aug. 13, 2009

(54) DIRECT HYDROCARBON AMINATION

(75) Inventors: Frederik Van Laar, Dubai (AE); Ekkehard Schwab, Neustadt (DE); Hartwig Voss, Frankenthal (DE); Joachim-Thierry Anders, Gönnheim (DE); Sven Crone, Limburgerhof (DE); Wolfgang Mackenroth, Bad Dürkheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/064,635

(22) PCT Filed: Aug. 21, 2006
(86) PCT No.: PCT/EP2006/065474

§ 371 Date: Feb. 25, 2008

(30) Foreign Application Priority Data
Aug. 30, 2005 (DE) 102005041140.1

(51) Int. Cl. C07C 209/02

(52) U.S. Cl. 564/408

(57) ABSTRACT

A process for aminating hydrocarbons comprises physically removing hydrogen from the reaction mixture.

The invention relates to a process for aminating, preferably directly aminating, hydrocarbons, preferably by reaction of hydrocarbons, more preferably benzene, with ammonia, in which hydrogen is physically removed from the reaction mixture. In particular, the invention relates to processes for aminating hydrocarbons, preferably by reacting aromatic hydrocarbons, more preferably benzene, with ammonia, especially according to the following reaction which is preferably catalyzed:

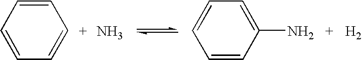

where hydrogen, especially the hydrogen formed in the amination, is removed from the reaction mixture by means of a hydrogen-permeable membrane.

The commercial preparation of amines, especially of aromatic amines such as aniline, is typically carried out in multistage reactions. Aniline is prepared, for example, typically by converting benzene to a benzene derivative, for example nitrobenzene, chlorobenzene or phenol, and subsequently converting this derivative to aniline.

More advantageous than such indirect processes for preparing especially aromatic amines are methods which enable direct preparation of the amines from the corresponding hydrocarbons. A very elegant route is the heterogeneously catalyzed direct amination of benzene, described for the first time in 1917 by Wibaut (Berichte, 50, 541-546). Since the direct amination is equilibrium-limited, several systems have been described which shift the equilibrium limitation by the selective removal of hydrogen from the reaction and enable increased benzene conversion. Most processes are based on the use of metal oxides which are reduced by hydrogen, hence remove the hydrogen from the reaction system and thus shift the equilibrium.

CN 1555921A discloses the oxide amination of benzene in the liquid phase, in which hydrogen peroxide functions as the “O” donor. However, the use of H₂O₂ is suitable for bulk chemicals only to a limited extent owing to the price and the low selectivity owing to subsequent reactions.

CA 553,988 discloses a process for preparing aniline from benzene, in which benzene, ammonia and gaseous oxygen are converted over a platinum catalyst at a temperature of about 1000° C. Suitable catalysts comprising platinum are platinum alone, platinum with certain specific metals and platinum together with certain specific metal oxides.

Moreover, CA 553,988 discloses a process for preparing aniline, in which benzene in the gas phase is reacted with ammonia in the presence of a reducible metal oxide at temperatures of from 100 to 1000° C., without addition of gaseous oxygen. Suitable reducible metal oxides are the oxides of iron, nickel, cobalt, tin, antimony, bismuth and copper.

U.S. Pat. No. 3,919,155 relates to the direct amination of aromatic hydrocarbons with ammonia, the catalyst used being a nickel/nickel oxide catalyst which may additionally comprise oxides and carbonates of zirconium, strontium, barium, calcium, magnesium, zinc, iron, titanium, aluminum, silicon, cerium, thorium, uranium and alkali metals.

U.S. Pat. No. 3,929,889 likewise relates to the direct amination of aromatic hydrocarbons with ammonia over a nickel/nickel oxide catalyst, the catalyst used having been reduced partly to elemental nickel and subsequently reoxidized in order to obtain a catalyst which has a ratio of nickel:nickel oxide of from 0.001:1 to 10:1.

U.S. Pat. No. 4,001,260 discloses a process for directly aminating aromatic hydrocarbons with ammonia, the catalyst used again being a nickel/nickel oxide catalyst which has been applied to zirconium dioxide and has been reduced with ammonia before use in the amination reaction.

U.S. Pat. No. 4,031,106 relates in turn to the direct amination of aromatic hydrocarbons with ammonia over a nickel/nickel oxide catalyst on a zirconium dioxide support which also comprises an oxide selected from lanthanides and rare earth metals.

DE 196 34 110 describes nonoxidative amination at a pressure of 10-500 bar and a temperature of 50-900° C., the reaction being effected in the presence of an acidic heterogeneous catalyst which has been modified with light and heavy platinum group metals.

WO 00/09473 describes a process for preparing amines by directly aminating aromatic hydrocarbons over a catalyst comprising at least one vanadium oxide.

WO 99/10311 teaches a process for directly aminating aromatic hydrocarbons at a temperature of <500° C. and a pressure of <10 bar. The catalyst used is a catalyst comprising at least one metal selected from transition metals, lanthanides and actinides, preferably Cu, Pt, V, Rh and Pd. To increase the selectivity and/or the conversion, preference is given to carrying out the direct amination in the presence of an oxidizing agent.

WO 00/69804 relates to a process for directly aminating aromatic hydrocarbons, the catalyst used being a complex comprising a noble metal and a reducible metal oxide. Particular preference is given to catalysts comprising palladium and nickel oxide or palladium and cobalt oxide.

All processes mentioned start from a mechanism for direct amination as detailed in the abstract of WO 00/69804. According to this, the desired amine compound is prepared first under (noble) metal catalysis from the aromatic hydrocarbon and ammonia, and, in a second step, the hydrogen formed in the first step is “scavenged” with a reducible metal oxide. The same mechanistic considerations form the basis of the process in WO 00/09473, in which the hydrogen is scavenged with oxygen from vanadium oxides (page 1, lines 30 to 33). The same mechanism is also the basis in U.S. Pat. No. 4,001,260, as is evident from the remarks and the figure in column 2, lines 16 to 44.

It is an object of the present invention to develop a particularly economical process for aminating hydrocarbons, especially for reacting benzene with ammonia, in which a continuous process is enabled with maximum selectivity.

This object is achieved by hydrogen being removed physically from the reaction mixture.

It has been found that, surprisingly, the conversion in the direct amination over metal catalysts (for example Ni, Fe, Co, Cu, NM or alloys thereof, where NM represents noble metals), compared with the equilibrium conversion, is increased substantially when the hydrogen formed in the reaction of the hydrocarbon with the ammonia is removed from the reaction mixture by use of hydrogen-permeable, preferably hydrogen-selective, membranes. Such membranes and also their production processes are known from the literature. Employment of this process allows aniline to be produced without deactivation over prolonged periods. In comparison to the addition of an exogenous gas, no complicated gas separation after reaction has to be carried out.

In the case of the known metal-metal oxide systems described at the outset, the cataloreactant has to be laden again with “oxygen” after a certain time. This means expensive interruptions since the amination and the reactivation typically do not proceed under the same conditions (pressure and temperature). The reaction thus typically has to be decompressed, flushed and inertized, and the catalyst reactivated and brought back under reaction conditions. The selectivity of the entire reaction changes with the oxygen content of the cataloreactant and thus has to be compensated for by process alteration (pressure, ammonia/aromatic ratio and/or temperature). Sufficient selectivity for the C and also N balance cannot be achieved with these systems, since ammonia is combusted by the metal oxides or by adsorbed oxygen on the surface to form N₂ and H₂O. In the case of addition of higher oxygen concentrations, ammonia can be converted not only to water and nitrogen but also to NOx. The hydrocarbons used too, especially the aromatics, can be combusted as a result of too high an oxygen concentration. Dilution allows the oxygen concentration to be controlled. However, this also means that the diluent component (typically N₂ in the case of air addition) has to be removed from the reaction mixture in a costly and inconvenient manner. The provision of a fully integrated solution is thus difficult to accomplish, if it can be accomplished at all, with metal oxide systems.

These disadvantages are avoided by the inventive physical removal of the hydrogen from the reaction system. The process according to the invention thus enables very efficient, selective, inexpensive direct amination.

The expression “physically remove” is understood to mean that the hydrogen escapes physically and preferably selectively from the reaction mixture. In comparison to known processes, in which the concentration of hydrogen in the reaction mixture is reduced by reaction, it is possible in accordance with the invention to dispense with the subsequent reaction and especially with the addition of components reactive toward hydrogen to the reaction system. Nor is it necessary for there to be any removal of these fundamentally undesired subsequent products from the actual process product, preferably the aniline.

Preference is given to physically removing the hydrogen from the reaction mixture by removing hydrogen from the reaction mixture by means of a hydrogen-permeable, preferably hydrogen-selective, membrane, preferably by virtue of the hydrogen diffusing out of the reaction mixture through the membrane. The diffusion of the hydrogen is preferably driven by the concentration gradient between the reaction system (retentate side) in which hydrogen is preferably formed by the reaction of benzene with ammonia, and the space on the other side of the membrane (permeate side). The hydrogen diffused to the permeate side can be depleted there, i.e. removed, preferably by being transported away, for example by means of gas flow or reduced pressure, and/or by chemical reaction, for example by reduction of an organic compound in the gas phase, for example benzene to cyclohexane, or with formation of water, preferably with an exogenous gas, for example air, preferably by catalyzed reaction with oxygen and/or air. This maintains or increases the concentration gradient between retentate side and permeate side, which drives the diffusion.

The hydrogen-permeable membrane may preferably be part of a reactor and, for example, may at least partly delimit the reaction chamber in which benzene is preferentially reacted with ammonia. The process according to the invention can preferably thus be effected such that the amination, preferably the reaction of benzene with ammonia, is effected in a membrane reactor with integrated hydrogen removal by means of a hydrogen-permeable membrane.

The membrane preferably has a permeance for hydrogen of greater than 10 m³/(m²×h×barⁿ), more preferably >50 m³/(m²×h×barⁿ), where n is theoretically 0.5 and actually between 0.5 and 0.6, and n is thus preferably between 0.5 and 0.6, more preferably n=0.5. The permeance (P) can be calculated from the hydrogen flow rate (in m³/(m²,h)) and the partial hydrogen pressures:

$$P = \frac{hydrogen flow rate}{{P}_{Retentate}^{n} - {P}_{Permeate}^{n}}$$

The membrane preferably has maximum selectivity for hydrogen. In other words, the membrane is preferably impervious and more preferably has an H₂/N₂ selectivity of >1000. The use of such membranes ensures that only a minimum fraction of reactant (hydrocarbon, ammonia) and/or product (especially aniline) passes to the permeate side of the membrane. For the preferred Pd and Pd-alloyed membranes, the reactants and hydrocarbons cannot diffuse through the membrane.

The membrane preferably has a thickness between 0.1 μm and 25 μm, more preferably between 0.5 μm and 10 μm, most preferably between 1 μm and 5 μm.

The membrane may have a self-supporting design. Owing to the generally very high material costs, it may be advantageous to fix the actual membrane on a porous ceramic and/or metallic supporting layer (“composite” membrane). This additionally offers the advantage that the membrane is stabilized and low layer thicknesses are also enabled. Typical membranes can be purchased, for example, from NGK in Japan or from Johnson Matthey.

Examples of suitable membranes include mesoporous inorganic membranes, microporous inorganic membranes, polymer membranes, membranes based on metals of transition group 4 or 5, coated with palladium, nanocrystalline metal membranes, mixed-conducting membranes and preferably membranes based on palladium or palladium alloys.

Mesoporous inorganic membranes are, for example, those having a pore size of less than 50 nm, for example those based on Al₂O₃.

Microporous inorganic membranes are, for example, those having a pore size of less than 2 nm, for example those based on ceramic or carbon molecular sieves. Useful ceramic molecular sieve membranes include zeolithic membranes, for example those of the MFI type (ZSM-5 or silicalite) which may if appropriate be supported on Al₂O₃, and amorphous membranes, for example those of SiO₂. Carbon molecular sieve membranes can be produced by carbonizing organic polymers, for example polyimide. Polymer membranes are “composite” membranes composed of an impervious hydrogen-selective polymer layer on an inorganic support.

Examples of useful membranes based on metals of transition group 4 and/or 5, which are coated with palladium, are those in which one or preferably two layers of palladium or palladium alloys are present on a nonporous support based on base metal, preferably vanadium, niobium and/or tantalum.

Also useful are membranes of TiN, nanocrystalline metal layers, for example Al₂O₃-supported palladium or ruthenium, or amorphous membranes.

Also suitable are mixed-conducting membranes which have both electronic and anionic conductivity.

However, preference is given to membranes which are based on palladium or palladium alloys. Useful alloys are especially alloys of palladium with silver and/or copper. Particular preference is given to membranes based on an alloy comprising palladium and between 23% by weight and 25% by weight of silver based on the total weight of the alloy. The alloy comprises more preferably between 75% by weight and 77% by weight of palladium based on the total weight of the alloy. Particular preference is also given to membranes based on an alloy comprising palladium and between 34% by weight and 46% by weight of copper based on the total weight of the alloy. The alloy comprises more preferably between 54% by weight and 66% by weight of palladium based on the total weight of the alloy. The palladium or the palladium alloy membranes may be doped further with rare earth metals, for example gadolinium. The palladium or the palladium alloy membranes may comprise typical further metals in customary amounts, which do not impair, or at least not significantly, the permeability and selectivity for hydrogen. These preferred membranes too may be present on porous substructures which fix and stabilize the actual membrane. The porous substructure may be based, for example, on ceramic, metal or an organic polymer, for example TiO₂ and/or Al₂O₃. The production of the preferably impervious metal membrane (preferably palladium or palladium alloy) on a porous support is common knowledge and can be effected, for example, by electroplating, sputtering, CVD (chemical vapor deposition) or preferably commonly known electroless wet-chemical coating.

Preferred membranes are additionally those which are catalytically active, and more preferably catalyze the reaction of H₂, especially with O₂, more preferably the amination of hydrocarbons, especially benzene with ammonia. The catalytically active layer may preferably be present in the substructure of the membrane.

As already explained, the membrane preferably separates the retentate side (reaction side) from the permeate side, the hydrogen formed on the retentate side passing through the membrane to the permeate side, where the hydrogen is removed by reaction, preferably reaction with oxygen or an exogenous stream, for example air, preferably in the presence of catalysts, and/or mass transfer, preferably by means of a gas stream (“sweep gas” or purge gas). The membrane preferably separates the retentate side (reaction side) from the permeate side, the hydrogen formed on the retentate side passing through the membrane to the permeate side, where the hydrogen is removed by chemical reaction which is catalyzed by a hydrogen oxidation catalyst, preferably by oxidation with oxygen. The selective removal of the hydrogen on the permeate side enables a distinct further reduction in the partial hydrogen pressure on the retentate side of the membrane (=reaction side) and thus enables the desired high benzene conversions (>5 mol %, >10 mol %, >20 mol % based on the amount of benzene added) with high aniline selectivity (>95%, >98%, >99%, aniline selectivity: mol of aniline/sum of all products formed in mol (benzene conversion)).

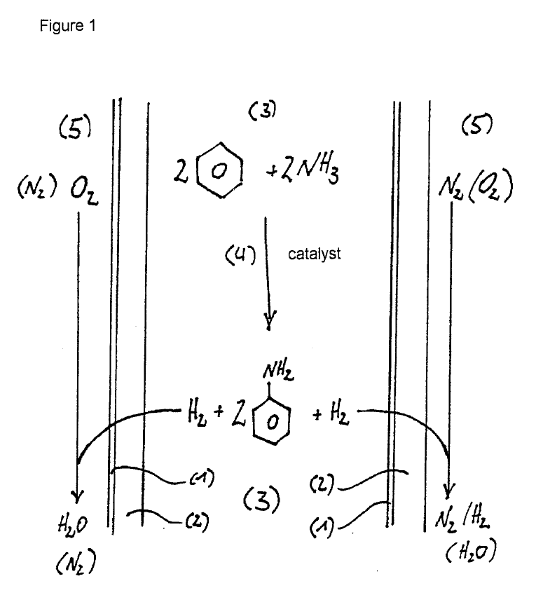

An example of an arrangement of a membrane (1) on a porous support (2) which separates a retentate side (3), into which benzene and ammonia are fed and preferably converted in the presence of catalyst (4), from the permeate side (5) is shown in FIG. 1. The membrane (1) may be arranged on the retentate side (see right-hand side of the figure) and/or the permeate side of the support material (2) (see left-hand side of the figure). The left-hand permeate side of FIG. 1 schematically shows the reaction of hydrogen with oxygen, preferably in the presence of catalyst, while the right-hand half depicts the transportation away, if appropriate additionally with reaction with oxygen.

The process according to the invention, i.e. the amination of hydrocarbons, especially the reaction of benzene with ammonia, can preferably be carried out in the presence of compounds which catalyze the amination.

The catalysts used may be the catalysts known for the direct amination of hydrocarbons, especially those known for the direct amination of benzene with ammonia to give aniline. Such catalysts have been described variously in the patent literature and are common knowledge. Since the hydrogen is removed in the process according to the invention by physical transport and not by chemical conversion in the reaction system, catalysts which do not have any components reactive toward hydrogen may also find use. Examples of useful catalysts include customary metal catalysts, for example those based on nickel, iron, cobalt, copper, noble metals or alloys of these metals mentioned. Useful noble metals (NM) may be all noble metals, for example Ru, Rh, Pd, Ag, Ir, Pt and Au, the noble metals Ru and Rh preferably not being used alone but rather in alloy with other transition metals, for example Co, Cu, Fe and nickel or mixtures thereof. Such alloys are also used with preference in the case of use of the other noble metals; for example, supported NiCuNM; CoCuNM; NiCoCuNM, NiMoNM, NiCrNM, NiReNM, CoMoNM, CoCrNM, CoReNM, FeCuNM, FeCoCuNM, FeMoNM, FeReNM alloys are of interest, where NM is a noble metal, especially preferably Ag and/or Au.

The catalyst may be used in generally customary form, for example as a powder or as a system useable in a fixed bed (for example extrudates, spheres, tablets, rings, in which case the catalytically active constituents may, if appropriate, be present on a support material. Examples of useful support materials include inorganic oxides, for example ZrO₂, SiO₂, Al₂O₃, MgO, TiO₂, B₂O₃, CaO, ZnO, BaO, ThO₂, CeO₂, Y₂O₃ and mixtures of these oxides, for example magnesium aluminum oxide, preferably TiO₂, ZrO₂, Al₂O₃, magnesium aluminum oxide and SiO₂, more preferably ZrO₂ and magnesium aluminum oxide. ZrO₂ is understood to mean both pure ZrO₂ and the normal Hf-comprising ZrO₂.

The catalysts used with preference in the process according to the invention may be regenerated, for example, by passing a reductive atmosphere (for example H₂ atmosphere) over the catalyst, or conducting first an oxidative atmosphere and then a reductive atmosphere over or through the catalyst bed.

It is possible with the amination process according to the invention to aminate any hydrocarbons, such as aromatic hydrocarbons, aliphatic hydrocarbons and cycloaliphatic hydrocarbons, which may have any substitution and may have heteroatoms and double or triple bonds within their chain or their ring/their rings. In the amination process according to the invention, preference is given to using aromatic hydrocarbons and heteroaromatic hydrocarbons. The particular products are the corresponding arylamines or heteroarylamines.

In the context of the present invention, an aromatic hydrocarbon is understood to be an unsaturated cyclic hydrocarbon which has one or more rings and comprises exclusively aromatic C—H bonds. The aromatic hydrocarbon preferably has one or more 5- or 6-membered rings.

A heteroaromatic hydrocarbon is understood to be those aromatic hydrocarbons in which one or more of the carbon atoms of the aromatic ring is/are replaced by a heteroatom selected from N, O and S.

The aromatic hydrocarbons or the heteroaromatic hydrocarbons may be substituted or unsubstituted. A substituted aromatic or heteroaromatic hydrocarbon is understood to be a compound in which one or more hydrogen atoms which is/are bonded to a carbon atom or heteroatom of the aromatic ring is/are replaced by another radical. Such radicals are, for example, substituted or unsubstituted alkyl, alkenyl, alkynyl, heteroalkyl, heteroalkenyl, heteroalkynyl, cycloalkyl and/or cycloalkynyl radicals. In addition, the following radicals are possible: halogen, hydroxyl, alkoxy, aryloxy, amino, amido, thio and phosphino. Preferred radicals of the aromatic or heteroaromatic hydrocarbons are selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido, where C₁₋₆ relates to the number of carbon atoms in the main chain of the alkyl radical, of the alkenyl radical or of the alkynyl radical, and C₃₋₈ to the number of carbon atoms of the cycloalkyl or cycloalkenyl ring. It is also possible that the substituents (radicals) of the substituted aromatic or heteroaromatic hydrocarbon have further substituents.

The number of substituents (radicals) of the aromatic or heteroaromatic hydrocarbon is arbitrary. In a preferred embodiment, the aromatic or heteroaromatic hydrocarbon has, however, at least one hydrogen atom which is bonded directly to a carbon atom or a heteroatom of the aromatic ring. Thus, a 6-membered ring preferably has 5 or fewer substituents (radicals) and a 5-membered ring preferably has 4 or fewer substituents (radicals). A 6-membered aromatic or heteroaromatic ring more preferably has 4 or fewer substituents, even more preferably 3 or fewer substituents (radicals). A 5-membered aromatic or heteroaromatic ring preferably bears 3 or fewer radicals, more preferably 2 or fewer radicals.

In a particularly preferred embodiment of the process according to the invention, an aromatic or heteroaromatic hydrocarbon of the general formula

(A)−(B)ₙ

is used, where the symbols are each defined as follows:
A is independently aryl or heteroaryl, A is preferably selected from phenyl, diphenyl, benzyl, dibenzyl, naphthyl, anthracene, pyridyl and quinoline;
n is from 0 to 5, preferably from 0 to 4, especially in the case when A is a 6-membered aryl or heteroaryl ring; in the case that A is a 5-membered aryl or heteroaryl ring, n is preferably from 0 to 4; irrespective of the ring size, n is more preferably from 0 to 3, most preferably from 0 to 2 and in particular from 0 to 1; the remaining carbon atoms or heteroatoms of A which do not bear any substituents B bear hydrogen atoms, or, if appropriate, no substituents; each
B is independently selected from the group consisting of alkyl, alkenyl, alkynyl, substituted alkyl, substituted alkenyl, substituted alkynyl, heteroalkyl, substituted heteroalkyl, heteroalkenyl, substituted heteroalkenyl, heteroalkynyl, substituted heteroalkynyl, cycloalkyl, cycloalkenyl, substituted cycloalkyl, substituted cycloalkenyl, halogen, hydroxy, alkoxy, aryloxy, carbonyl, amino, amido, thio and phosphino; B is preferably independently selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido.

The term “independently” means that, when n is 2 or greater, the substituents B may be identical or different radicals from the groups mentioned.

In the present application, alkyl is understood to mean branched or unbranched, saturated acyclic hydrocarbyl radicals. Examples of suitable alkyl radicals are methyl, ethyl, n-propyl, i-propyl, n-butyl, t-butyl, i-butyl, etc. The alkyl radicals used preferably have from 1 to 50 carbon atoms, more preferably from 1 to 20 carbon atoms, even more preferably from 1 to 6 carbon atoms and in particular from 1 to 3 carbon atoms.

In the present application, alkenyl means branched or unbranched, acyclic hydrocarbyl radicals which have at least one carbon-carbon double bond. Suitable alkenyl radicals are, for example, 2-propenyl, vinyl, etc. The alkenyl radicals have preferably from 2 to 50 carbon atoms, more preferably from 2 to 20 carbon atoms, even more preferably from 2 to 6 carbon atoms and in particular from 2 to 3 carbon atoms. The term alkenyl also encompasses radicals which have either a cis-orientation or a trans-orientation (alternatively E or Z orientation).

In the present application, alkynyl is understood to mean branched or unbranched, acyclic hydrocarbyl radicals which have at least one carbon-carbon triple bond. The alkynyl radicals preferably have from 2 to 50 carbon atoms, more preferably from 2 to 20 carbon atoms, even more preferably from 1 to 6 carbon atoms and in particular from 2 to 3 carbon atoms.

Substituted alkyl, substituted alkenyl and substituted alkynyl are understood to mean alkyl, alkenyl and alkynyl radicals in which one or more hydrogen atoms which are bonded to one carbon atom of these radicals are replaced by another group. Examples of such other groups are heteroatoms, halogen, aryl, substituted aryl, cycloalkyl, cycloalkenyl, substituted cycloalkyl, substituted cycloalkenyl and combinations thereof. Examples of suitable substituted alkyl radicals are benzyl, trifluoromethyl, inter alia.

The terms heteroalkyl, heteroalkenyl and heteroalkynyl refer to alkyl, alkenyl and alkynyl radicals in which one or more of the carbon atoms in the carbon chain is replaced by a heteroatom selected from N, O and S. The bond between the heteroatom and a further carbon atom may be saturated, or, if appropriate, unsaturated.

In the present application, cycloalkyl is understood to mean saturated cyclic nonaromatic hydrocarbyl radicals which are composed of a single ring or a plurality of fused rings. Suitable cycloalkyl radicals are, for example, cyclopentyl, cyclohexyl, cyclooctanyl, bicyclooctyl, etc. The cycloalkyl radicals have preferably between 3 and 50 carbon atoms, more preferably between 3 and 20 carbon atoms, even more preferably between 3 and 8 carbon atoms and in particular between 3 and 6 carbon atoms.

In the present application, cycloalkenyl is understood to mean partly unsaturated, cyclic nonaromatic hydrocarbyl radicals which have a single fused ring or a plurality of fused rings. Suitable cycloalkenyl radicals are, for example, cyclopentenyl, cyclohexenyl, cyclooctenyl, etc. The cycloalkenyl radicals have preferably from 3 to 50 carbon atoms, more preferably from 3 to 20 carbon atoms, even more preferably from 3 to 8 carbon atoms and in particular from 3 to 6 carbon atoms.

Substituted cycloalkyl and substituted cycloalkenyl radicals are cycloalkyl and cycloalkenyl radicals, in which one or more hydrogen atoms of any carbon atom of the carbon ring is replaced by another group. Such other groups are, for example, halogen, alkyl, alkenyl, alkynyl, substituted alkyl, substituted alkenyl, substituted alkynyl, aryl, substituted aryl, cycloalkyl, cycloalkenyl, substituted cycloalkyl, substituted cycloalkenyl, an aliphatic heterocyclic radical, a substituted aliphatic heterocyclic radical, heteroaryl, substituted heteroaryl, alkoxy, aryloxy, boryl, phosphino, amino, silyl, thio, seleno and combinations thereof. Examples of substituted cycloalkyl and cycloalkenyl radicals are 4-dimethylaminocyclohexyl, 4,5-dibromocyclohept-4-enyl, inter alia.

In the context of the present application, aryl is understood to mean aromatic radicals which have a single aromatic ring or a plurality of aromatic rings which are fused, joined via a covalent bond or joined by a suitable unit, for example a methylene or ethylene unit. Such suitable units may also be carbonyl units, as in benzophenol, or oxygen units, as in diphenyl ether, or nitrogen units, as in diphenylamine. The aromatic ring or the aromatic rings are, for example, phenyl, naphthyl, diphenyl, diphenyl ether, diphenylamine and benzophenone. The aryl radicals preferably have from 6 to 50 carbon atoms, more preferably from 6 to 20 carbon atoms, most preferably from 6 to 8 carbon atoms.

Substituted aryl radicals are aryl radicals in which one or more hydrogen atoms which are bonded to carbon atoms of the aryl radical are replaced by one or more other groups. Suitable other groups are alkyl, alkenyl, alkynyl, substituted alkyl, substituted alkenyl, substituted alkynyl, cycloalkyl, cycloalkenyl, substituted cycloalkyl, substituted cycloalkenyl, heterocyclo, substituted heterocyclo, halogen, halogen-substituted alkyl (e.g. CF₃), hydroxyl, amino, phosphino, alkoxy, thio and both saturated and unsaturated cyclic hydrocarbyl radicals which may be fused on the aromatic ring or on the aromatic rings or may be joined by a bond, or may be joined to one another via a suitable group. Suitable groups have already been mentioned above.

According to the present application, heterocyclo is understood to mean a saturated, partly unsaturated or unsaturated, cyclic radical in which one or more carbon atoms of the radical are replaced by a heteroatom, for example N, O or S. Examples of heterocyclo radicals are piperazinyl, morpholinyl, tetrahydropyranyl, tetrahydrofuranyl, piperidinyl, pyrrolidinyl, oxazolinyl, pyridyl, pyrazyl, pyridazyl, pyrimidyl.

Substituted heterocyclo radicals are those heterocyclo radicals in which one or more hydrogen atoms which are bonded to one of the ring atoms are replaced by another group. Suitable other groups are halogen, alkyl, substituted alkyl, aryl, substituted aryl, heteroaryl, substituted heteroaryl, alkoxy, aryloxy, boryl, phosphino, amino, silyl, thio, seleno and combinations thereof.

Alkoxy radicals are understood to be radicals of the general formula —OZ¹ in which Z¹ is selected from alkyl, substituted alkyl, cycloalkyl, substituted cycloalkyl, heterocycloalkyl, substituted heterocycloalkyl, silyl and combinations thereof. Suitable alkoxy radicals are, for example, methoxy, ethoxy, benzyloxy, t-butoxy, etc. The term aryloxy is understood to mean those radicals of the general formula —OZ¹ in which Z¹ is selected from aryl, substituted aryl, heteroaryl, substituted heteroaryl and combinations thereof. Suitable aryloxy radicals are phenoxy, substituted phenoxy, 2-pyridinoxy, 8-quinolinoxy, inter alia.

Amino radicals are understood to be radicals of the general formula —NZ¹Z² in which Z¹ and Z² are each independently selected from hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof.

Aromatic or heteroaromatic hydrocarbons used with preference in the amination process according to the invention are selected from benzene, naphthalene, diphenylmethane, anthracene, toluene, xylene, phenol and aniline, and also pyridine, pyrazine, pyridazine, pyrimidine and quinoline. It is also possible to use mixtures of the aromatic or heteroaromatic hydrocarbons mentioned. Particular preference is given to using the aromatic hydrocarbons, benzene, naphthalene, anthracene, toluene, xylene, pyridine, phenol and aniline, very particular preference to using benzene, toluene and pyridine.

Especially preferably, benzene is used in the amination process according to the invention, so that the product formed is aniline.

The compound through which the aminogroup is introduced is more preferably ammonia. This means that, in accordance with the invention, the hydrocarbons, especially the benzene, are more preferably reacted with ammonia. If appropriate, compounds which eliminate ammonia under the reaction conditions may also find use.

For the preparation of mono- and di-alkyl-N,(N)-substituted aromatic amines, for example mono- and/or dimethylaniline, it is also possible to use mono- and dialkylamines, preferably mono- and di(m)ethylamine.

The reaction conditions in the amination processes according to the invention are dependent upon factors including the aromatic hydrocarbon to be aminated and the catalyst used.

The amination, preferably the amination of benzene, i.e. the reaction of benzene with ammonia, is effected generally at temperatures of from 200 to 800° C., preferably from 300 to 700° C., more preferably from 325 to 600° C. and most preferably from 350 to 500° C.

The reaction pressure in the amination, preferably in the amination of benzene, i.e. the reaction of benzene with ammonia, is generally from 1 to 900 bar, preferably from 1 to 300 bar, in particular from 5 to 125 bar, especially preferably from 15 to 110 bar.

The residence time in the amination process according to the invention, preferably in the amination of benzene, is generally from 15 minutes to 8 hours, preferably from 15 minutes to 4 hours, more preferably from 15 minutes to 1 hour, in the case of performance in a batchwise process. In the case of performance in a preferred continuous process, the residence time is generally from 0.1 second to 20 minutes, preferably from 0.5 second to 10 minutes. For the preferred continuous processes, “residence time” in this context means the residence time over the catalyst, hence the time in the catalyst bed for fixed bed catalysts; for fluidized bed reactors, the synthesis part of the reactor (part of the reactor where the catalyst is localized) is considered.

The relative amount of the hydrocarbon used and of the amine component is dependent upon the amination reaction carried out and the reaction conditions. In general, at least stoichiometric amounts of the hydrocarbon and the amine component are used. However, it is typically preferred to use one of the reaction partners in a stoichiometric excess in order to achieve a shift in the equilibrium to the side of the desired product and hence a higher conversion. Preference is given to using the amine component in a stoichiometric excess.

The amination process according to the invention may be carried out continuously, batchwise or semicontinuously. Suitable reactors are thus both stirred tank reactors and tubular reactors. Typically reactors are, for example, high pressure stirred tank reactors, autoclaves, fixed bed reactors, fluidized bed reactors, moving beds, circulating fluidized beds, salt bath reactors, plate heat exchangers as reactors, tray reactors having a plurality of trays with or without heat exchange or drawing/feeding of substreams between the trays, in possible designs as radial flow or axial flow reactors, continuous stirred tanks, bubble reactors, etc., and the reactor suitable in each case for the desired reaction conditions (such as temperature, pressure and residence time) is used. The reactors may each be used as a single reactor, as a series of individual reactors and/or in the form of two or more parallel reactors. The reactors may be operated in an AB mode (alternating mode). The process according to the invention may be carried out as a batch reaction, semicontinuous reaction or continuous reaction. The specific reactor construction and performance of the reaction may vary depending on the amination process to be carried out, the state of matter of the aromatic hydrocarbon to be aminated, the required reaction times and the nature of the nitrogen-containing catalyst used. Preference is given to carrying out the process according to the invention for direct amination in a high pressure stirred tank reactor, fixed bed reactor or fluidized bed reactor.

In a particularly preferred embodiment, a fixed bed or fluidized bed reactor is used in the amination of benzene to aniline, in which case the membranes are arranged internally and hydrogen is thus removed in the synthesis part. A further advantage of the membrane, which can be flowed through by means of a purge stream, is good thermal control of the reactor: heat of reaction can be added or preferably removed by heating or cooling the purge stream.

The hydrocarbon and the amine component may be introduced in gaseous or liquid form into the reaction zone of the particular reactor. The preferred phase is dependent in each case upon the amination carried out and the reactor used. In a preferred embodiment, for example in the preparation of aniline from benzene, benzene and ammonia are preferably present as gaseous reactants in the reaction zone. Typically, benzene is fed as a liquid which is heated and evaporated to form a gas, while ammonia is present either in gaseous form or in a supercritical phase in the reaction zone. It is likewise possible that benzene is present in a supercritical phase at least together with ammonia.

The hydrocarbon and the amine component may be introduced together into the reaction zone of the reactor, for example as a premixed reactant stream, or separately. In the case of a separate addition, the hydrocarbon and the amine component may be introduced simultaneously, offset in time or successively into the reaction zone of the reactor. Preference is given to adding the amine component and adding the hydrocarbon offset in time.

If appropriate, further coreactants, cocatalysts or further reagents are introduced into the reaction zone of the reactor in the process according to the invention, depending in each case on the amination carried out. For example, in the amination of benzene, oxygen or an oxygen-comprising gas may be introduced into the reaction zone of the reactor. The relative amount of gaseous oxygen which can be introduced into the reaction zone is variable and depends upon factors including the catalyst system used. The molar ratio of gaseous oxygen to aniline may, for example, be in the range from 0.05:1 to 1:1, preferably from 0.1:1 to 0.5:1. However, it is also possible to carry out the amination of benzene without addition of oxygen or an oxygen-comprising gas into the reaction zone. Preference is given to adding the oxygen-comprising gas on the permeate side of the membrane and oxidizing hydrogen catalytically to water there. As this is done, the hydrogen concentration on the permeate side is preferably kept as close as possible to zero.

After the amination, the desired product can be isolated by processes known to those skilled in the art.

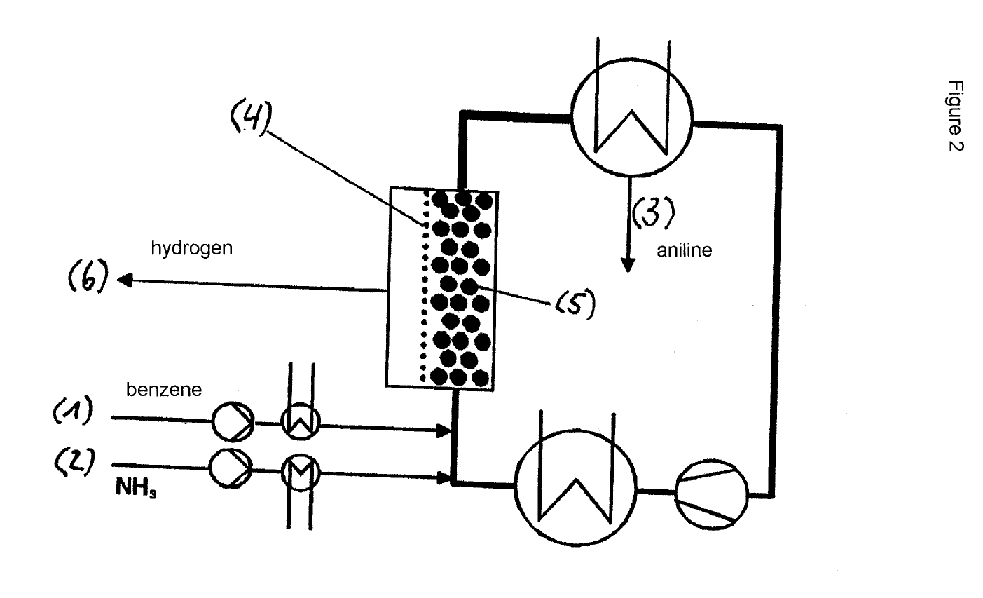

A preferred process scheme is outlined in FIG. 2. In this figure, the reference numerals are defined as follows:
1: Benzene fed
2: Ammonia fed
3: Removal of aniline, for example after partial condensation
4: Membrane
5: Catalyst
6: Removal of hydrogen by chemical conversion and/or being transported away

The process according to the invention for preparing aniline can thus more preferably be effected by reacting benzene with ammonia in such a way that a reaction mixture into which benzene and ammonia are fed separately or together and preferably continuously is conducted in a circulation system which has a membrane reactor with catalyst and hydrogen-permeable membrane, the pressure of the reaction mixture comprising benzene and ammonia in the presence of catalyst in the membrane reactor is between 10 and 150 bar, and aniline is preferably removed continuously from the circulation system, preferably after condensation of the aniline. The hydrogen which diffuses through the membrane, on the permeate side of the membrane reactor, i.e. on the side of the membrane facing away from the reaction mixture, is preferably transported away, for example by means of reduced pressure or preferably by means of a gas stream, for example air or nitrogen, and/or by chemical conversion, preferably by reaction with oxygen or air. The transmembrane pressure is preferably adjusted to a high level (ΔP preferably between 1 and 100 bar). However, the transmembrane pressure that can be established is limited by the mechanical stability of the membrane; this results in a transmembrane pressure for Pd or palladium alloy on ceramic “composite” membranes of typically between 5 and 50 bar.

1. A process for aminating a hydrocarbon, which comprises physically removing hydrogen from the reaction mixture.

2. A process for aminating a hydrocarbon, which comprises removing hydrogen from the reaction mixture by means of a hydrogen-permeable membrane.

3. The process according to claim 2, wherein the amination is effected in a membrane reactor with integrated hydrogen removal by means of a hydrogen-permeable membrane.

4. The process according to claim 2, wherein the membrane has a permeance for hydrogen of greater than 10 m³/(m²×h×bar^{0.5}).

5. The process according to claim 2, wherein the membrane is based on palladium.

6. The process according to claim 2, wherein the membrane is based on palladium alloys.

7. The process according to claim 6, wherein the membrane is based on an alloy comprising palladium and between 23% by weight and 25% by weight of silver based on the total weight of the alloy.

8. The process according to claim 6, wherein the membrane is based on an alloy comprising palladium and between 34% by weight and 46% by weight of copper based on the total weight of the alloy.

9. The process according to claim 2, wherein the membrane has a thickness between 0.1 μm and 25 μm.

10. The process according to claim 2, wherein the membrane is fixed on a porous ceramic and/or metallic supporting layer.

11. The process according to claim 2, wherein the membrane separates the retentate side (reaction side) from the permeate side so that the hydrogen formed on the retentate side passes through the membrane to the permeate side, where the hydrogen is removed by reaction and/or mass transfer.

12. The process according to claim 2, wherein the membrane separates the retentate side (reaction side) from the permeate side so that the hydrogen formed on the retentate side passes through the membrane to the permeate side, where the hydrogen is removed by chemical reaction which is catalyzed by a hydrogen oxidation catalyst.

13. The process according to claim 1, wherein the amination of the hydrocarbon is catalyzed.

14. The process according to claim 1, wherein the amination is carried out at temperatures between 200 and 800° C.

15. The process according to claim 1, wherein the amination is carried out at pressures between 1 and 900 bar.

16. The process for preparing aniline according to claim 1, wherein a reaction mixture into which benzene and ammonia are fed separately or together is conducted in a circulation system which includes a membrane reactor with a catalyst and a hydrogen-permeable membrane, in which the pressure of the reaction mixture comprising benzene and ammonia in the presence of the catalyst in the membrane reactor is between 10 and 150 bar, and aniline is removed from the circulation system.

17. The process according to claim 1, wherein the hydrocarbon is an aromatic hydrocarbon of the formula
(A)−(B)ₙ
in which the symbols are each defined as follows:
A is independently aryl or heteroaryl
n is from 0 to 5 and
each B is independently selected from the group consisting of alkyl, alkenyl, alkynyl, substituted alkyl, substituted alkenyl, substituted alkynyl, heteroalkyl, heteroalkenyl, heteroalkynyl, substituted heteroalkyl, substituted heteroalkenyl, substituted heteroalkynyl, cycloalkyl, cycloalkenyl, substituted cycloalkyl, substituted cycloalkenyl, halogen, hydroxy, alkoxy, aryloxy, carbonyl, amino, amido, thio and phosphino.

18. The process according to claim 1, wherein the hydrocarbon is benzene.
